(19) United States
(12) Patent Application Publication
(10) Pub. No.: US 2005/0205098 A1
(43) Pub. Date: Sep. 22, 2005

(54) Apparatus and method to deliver dilute O2 by nasal cannula or facemask

(76) Inventors: Samsun Lampotang, Gainesville, FL (US); Nikolaus Gravenstein, Gainesville, FL (US)

Correspondence Address: AKERMAN SENTERFITT, P.O. BOX 3188, WEST PALM BEACH, FL, 33402-3188, US

(21) Appl. No.: 11/084,365

(22) Filed: Mar. 18, 2005

Related U.S. Application Data
(60) Provisional application No. 60/554,897, filed on Mar. 19, 2004.

(51) Int. Cl. A62B 7/00; A61M 16/00

(52) U.S. Cl. 128/207.18; 128/204.18

(57) ABSTRACT

An apparatus for delivering an O₂ comprising gas mixture by nasal cannula or face mask to a patient includes at least one jet pump having an inlet for receiving a high pressure O₂ rich flow and an outlet for emitting a reduced O₂ content flow as compared to the O₂ rich flow at its inlet. The jet pump includes at least one air entrainment aperture for entraining room air and provides the reduced O₂ content gas flow at its outlet, thus providing O₂ dilution. A nasal cannula or face mask is in fluid connection with the outlet of the jet pump. The nasal cannula or face mask includes a fluid conduit terminating at a pair of apertured nostril outlet prongs or face facemask, respectively, for providing the reduced O₂ content gas flow to the patient.

### CROSS-REFERENCE TO RELATED APPLICATIONS

This application claims the benefit of U.S. Provisional Patent Application No. 60/554,897 entitled “APPARATUS AND METHOD TO DELIVER DILUTE O₂ BY NASAL CANNULA OR FACEMASK” filed on Mar. 19, 2004, which is hereby incorporated by reference into the present application in its entirety.

### STATEMENT REGARDING FEDERALLY SPONSORED RESEARCH OR DEVELOPMENT

Not applicable.

### FIELD OF THE INVENTION

The invention relates to medical appliances. More specifically, the invention relates to apparatus and methods for delivering an O₂ comprising gas mixture by nasal cannula or facemask.

### BACKGROUND

Nasal cannula assemblies and facemasks have found widespread use to provide supplemental O₂ or other gases to patients for use over a relatively long period of time. Nasal cannulae have largely replaced O₂ masks and provide much greater comfort than nasal catheters. The use of such devices has proved sufficiently beneficial so that they are widely used not only by respiratory patients, but also for a wide variety of patients who benefit from the O₂ added by such assemblies.

The most commonly used arrangement includes a dual prong nose piece which is centered in a loop of vinyl tubing. The nose piece openings are inserted in the nose with the tubing tucked behind the ears.

Pure O₂ is generally delivered to patients via nasal cannulae. However, such a practice can pose fire risks. The Joint Commission on Accreditation of Healthcare Organizations (JCAHO) has recently issued a sentinel event alert about operating room fires and has recommended the use of about 30 volume percent O₂ or less when supplying supplemental O₂ via nasal cannula or other open delivery systems, such as face masks or face tents.

It is estimated that 50 to 100 surgical fires occur in the United States every year. The presence of an O₂-enriched atmosphere has been estimated to be involved in about 74% of all surgical fires.

Three elements are required for the classic fire triad. An ignition source (e.g., a spark), an oxidizer (e.g., free-flowing O₂), and fuel (e.g., bedding, drapes, patient hair, patient cap, patient clothing, skin prep solution, or sponges). A fire may occur when these three elements are present under the right conditions. Keeping these elements apart helps prevent surgical fires and protects the patient from injury, but separation of the various fire triad components is not always possible, practical or fully effective.

### SUMMARY

An apparatus for delivering an O₂ comprising gas mixture by nasal cannula or face mask to a patient includes at least one jet pump (also known as an ejector or “venturi”) having an inlet for receiving a high pressure O₂ rich flow and an outlet for emitting a reduced O₂ content flow as compared to the O₂ rich flow at its inlet. The jet pump includes at least one air entrainment aperture for entraining room air and an outlet for providing the reduced O₂ content gas flow. A nasal cannula or face mask is in fluid connection with the outlet of the jet pump. The nasal cannula or facemask includes a fluid conduit terminating at a pair of apertured nostril outlet prongs or face facemask, respectively, for providing the reduced O₂ content gas flow to the patient. By providing an O₂ gas mixture having significant dilution, the invention significantly reduces the risk of surgical fires.

The jet pump can be integrally formed with the nasal cannula or face mask, or be removably coupled thereto. In the removable embodiment, the nasal cannula or face mask is disposable and the jet pump is reusable. The apparatus can include structure for adjusting an effective area of the entrainment aperture(s).

In one embodiment, a switch is disposed between a source of the high pressure O₂ rich flow and the inlet of the jet pump. When this switch is open the jet pump becomes directly fluidly connected to the high pressure O₂ rich source.

An O₂ sensor can be disposed in fluid communication with the jet pump outlet. The sensor provides a measured percentage of O₂ in the reduced O₂ content gas flow and generates an electrical output signal based on the measured percentage. Interface circuitry including an A/D converter is preferably provided in this embodiment for converting the electrical output signal to a digital signal. A microprocessor-based controller including a memory which stores an O₂ set point can be provided for receiving the digital signal, wherein the controller compares the measured percentage to the O₂ set point and generates control signals in response to the comparing. An oxygen valve can be provided for controlling a flow of high pressure O₂ rich flow in series with the input of the jet pump, wherein the driver circuitry is communicably connected to at least one of the jet pump and the oxygen valve. Responsive to the control signals, the driver circuitry sends adjustment signals to the oxygen valve or jet pump so that the reduced O₂ content gas flow tracks the O₂ set point.

A related method for delivering a dilute O₂ comprising gas mixture by nasal cannula or face mask to a patient includes the steps of providing a high pressure O₂ rich flow, entraining room air using the O₂ rich flow using at least one jet pump to provide a reduced O₂ content gas flow, and delivering the reduced O₂ content gas flow via a nasal cannula or face mask to a patient.

### BRIEF DESCRIPTION OF THE DRAWINGS

A fuller understanding of the present invention and the features and benefits thereof will be accomplished upon review of the following detailed description together with the accompanying drawings, in which:

FIG. 1 shows a flow apparatus including a jet pump removably connected to a nasal cannula, according to an embodiment of the invention.

FIG. 2 shows a jet pump mixing chamber having a plurality of entrainment port apertures along with a sliding dial which can modify the area of apertures for entraining ambient air, according to an embodiment of the invention.

FIG. 3 shows a supplemental O₂ delivery system including a jet pump removably connected to a nasal cannula or face mask, along with an O₂ flow sensor and microprocessor, according to another embodiment of the invention.

### DETAILED DESCRIPTION

An apparatus for delivering an O₂ comprising gas mixture by nasal cannula or face mask to a patient includes at least one jet pump having an inlet for receiving a high pressure O₂ rich flow and an outlet for emitting a reduced O₂ content flow as compared to the O₂ rich flow at its inlet. The jet pump includes at least one air entrainment aperture for entraining room air and provides the reduced O₂ content gas flow at its outlet, thus providing O₂ dilution. A nasal cannula or face mask is in fluid connection with the outlet of the jet pump. The nasal cannula or face mask includes a fluid conduit terminating at a pair of apertured nostril outlet prongs or face facemask, respectively, for providing the reduced O₂ content gas flow to the patient.

Although the invention will have significant utility in the operating room, the invention is in no way limited to use in the operating room. The invention can be used more generally for procedures where some type of cautery is in use and/or less than 100% O₂ fulfills applicable clinical and safety considerations.

Jet pumps according to the invention are preferred over electromechanical pumps. Jet pumps do not require electrical energy to operate, have no moving parts and provide high reliability and essentially unlimited time in operation. Jet pumps according to the invention are driven entirely by the potential energy of the gas from a pressurized O₂ comprising source. Although jet pumps are preferred, other pump types may be used with the invention.

Jet pumps include various components designed to control pressure/flow characteristics. These include a high-speed gas ejection nozzle, a stream mixing chamber with diffuser and a receiving chamber for further gas mixing. Gas passing through the nozzle forms a high-velocity stream in the receiving chamber. This high-speed stream generates a lower pressure region at its boundary (according to the Bernoulli principle) and thereby aspirates gas from a fluidly connected gas source, such as ambient air in the case of the preferred embodiment of the invention. The two streams of gas (air and O₂) are directed into the mixing chamber where their speed is equalized due to the mixing. The mixed stream then passes through a diffuser, where the stream is expanded, and the static pressure increases.

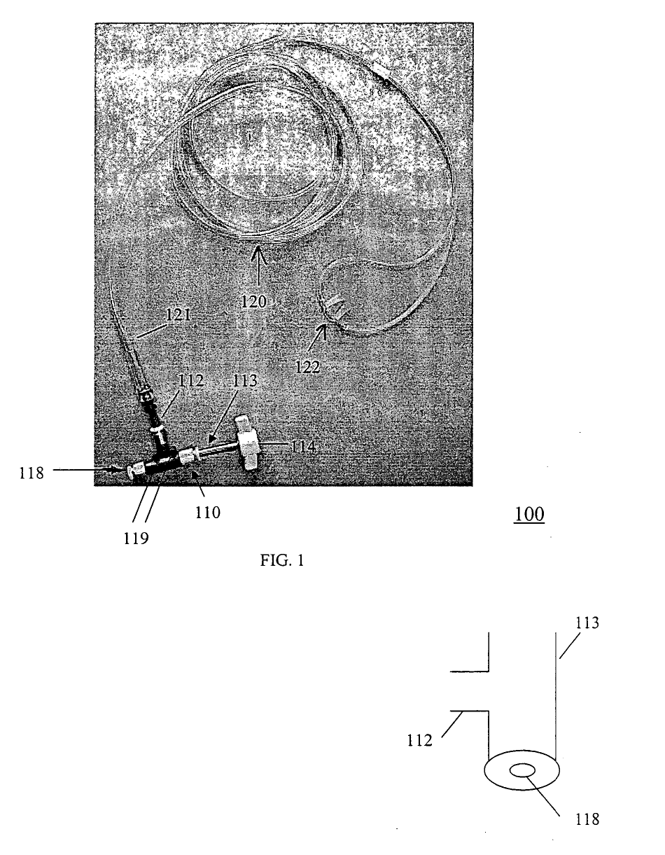

Referring to FIG. 1, a first embodiment of the invention is shown. Apparatus 100 delivers an O₂ comprising gas mixture by nasal cannula to a patient (not shown). Apparatus 100 includes a jet pump 110, such as the Pisco VUL 07 provided by PISCO USA, INC. Bensenville, Ill., remotely located from the patient (not shown). A nasal cannula 120 is fluidly connected to an outlet 112 of the jet pump. Although a nasal cannula 120 is shown fluidicly connected to jet pump 110, those having ordinary skill in the art will appreciate that jet pump 110 can be generally connected to any open oxygen comprising delivery system, such as a face mask or face tent. Jet pump inlet 113 includes wing nut 114 which is used to connect to the output of a standard ball-in-tube flow meter (not shown) which supplies a high pressure O₂ flow. A flow meter (not shown) is generally connected to a pressurized source of pure O₂ (not shown), such as at about 50 psi.

The jet pump 110 includes at least one air entrainment aperture 118 for entraining room air and providing a reduced O₂ content gas flow at outlet 112. The inset in the lower right corner of FIG. 1 more clearly shows an exemplary entrainment aperture 118. Jet pump 110 shown in FIG. 1 also includes mounting holes 119 for secure installation, the holes 119 accommodating fasteners such as screws.

Nasal cannula 120 includes a fluid conduit comprising a vinyl tube 121 (typical internal diameter of less than about ¼ inch) terminating at a pair of apertured nostril outlet prongs 122 for providing the reduced O₂ content gas flow a patient. The reduced O₂ gas mixture provided which has significant dilution significantly reduces the risk of surgical fires. The reduced O₂ gas mixture can range from about 21% (the O₂% of ambient air) to nearly 100%. The reduced O₂ gas mixture can be adjusted, including dynamic adjustment, such as by modifying the size of the air entrainment port 118, and/or changing the O₂ pressure applied to the inlet 114 of jet pump 110. Depending on the desired application, the reduced ° 2 gas mixture can be 21% (pure air), 25%, 30%, 35%, 40%, 45%, 50%, 55%, 60%, 65%, 70%, 80%, 90% to 100% (air entrainment aperture 118 fully closed).

The flow rate provided by nasal cannula or other open delivery system according to the invention is generally 2 to 5 lpm total flow rate. However, flows higher or lower than this range can be provided by altering the oxygen supply pressure, the aperture of the nozzle that delivers the high pressure O₂, the flow rate of high pressure oxygen delivered to the jet pump 10 and the entrainment ratio.

Although nasal cannula 120 is shown in FIG. 1 as being removably connected to jet pump 110 and thus disposable, the jet pump 110 can be integrally formed with the nasal cannula to form a unitary apparatus 100. For example, in this embodiment, apparatus 100 can be formed from a single piece of molded plastic, jet pump and tubing welded together, or other unitary arrangements. The air entrainment device, such as jet pump 110, is preferably located remote to the patient. This ensures that the delivered gas in the immediate vicinity of the patient even if still in its delivery conduit is already diluted down. This remote arrangement significantly reduces the likelihood that an accidental leak in the gas delivery conduit or inadvertent exposure of the delivery tubing to a laser beam or cautery unit will result in a surgical fire.

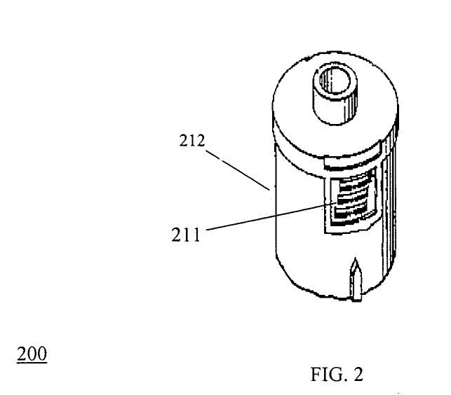

As noted above, the amount of O₂ dilution can be modified. For example, jet pump 110 can include structure for adjusting a size of aperture 113, such as a sliding gate, or even tape or the finger of a person. FIG. 2 shows a jet pump mixing chamber 200 having a plurality of entrainment apertures 211 along with a sliding dial 212 which can modify the effective area of apertures 211, the effective area being the area exposed to the ambient.

When added dilution is desired, the flow meter generally used to supply O₂ to the jet pump can be bypassed so that the jet pump is directly fluidly connected to the high pressure O₂ supply (not shown). In this embodiment, a switch (not shown) is generally interposed between the supply and jet pump 110 to permit shutting off the flow to the jet pump.

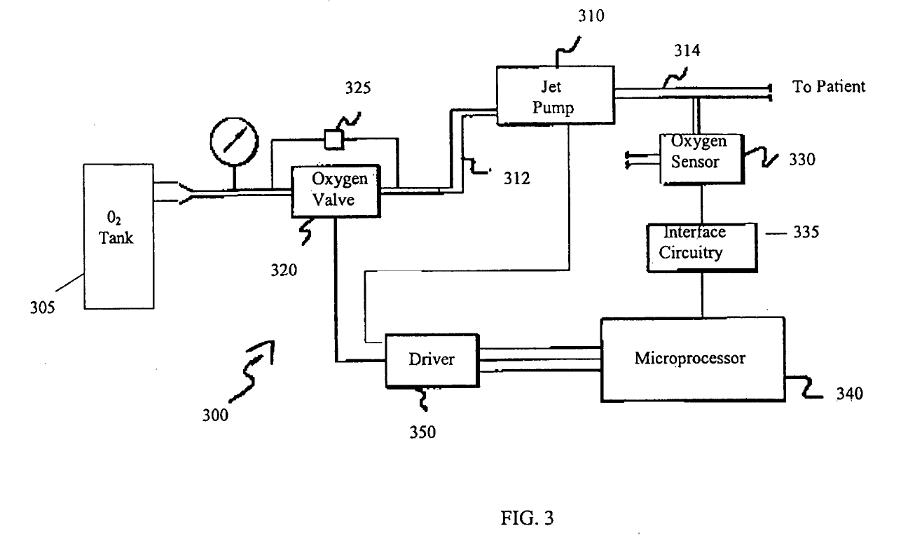

In one embodiment of the invention, an O₂ sensor or gas sampling site can be disposed in fluid communication with the outlet of jet pump 110. FIG. 3 shows a supplemental O₂ delivery system 300 including a jet pump removably connected to a nasal cannula, along with an O₂ flow sensor and microprocessor, according to another embodiment of the invention.

System 300 includes O₂ supply tank 305, O₂ valve 320 and bypass valve 325 which is normally off. Conduit 312 provides ° 2 gas to the input of jet pump 310 (entrainment aperture(s) not shown). Cannula 314 is attached to the output of jet pump 310 and delivers the reduced O₂ gas mixture to a patient.

An O₂ sensor 330 samples or measures the percentage of O₂ exiting from the outlet of jet pump 310 near the input of cannula 314 and generates an electrical output based on the measured percent composition of O₂. The O₂ sensor may be located remote from jet pump 310 and include a small pump (not shown) for aspirating a gas sample exiting jet pump 310. The aspiration is preferably, but not necessarily, continuous. The sensor output is amplified, filtered and A/D converted by interface circuitry 335 and is then forwarded to microprocessor-based controller 340. Controller 340 includes a memory which stores an O₂ set point, which is the level that a clinician generally sets. Controller 340 compares the output of the O₂ sensor 330 to the set point level of O₂. The controller 340 generates a response signal based on the comparison, which is communicated to the driver circuit 350, described below.

In the preferred embodiment, flow valve 320 defines a passage (not shown) through which a gas traverses and a flow controlling structure for adjusting the passage to change the rate of flow of the gas therethrough. The feedback circuit comprising O₂ sensor 330, interface circuitry 335, controller 340 and driver circuit 350 adjusts the flow provided by flow valve 320, if necessary, so that the percentage composition of the desired gas exiting the jet pump 310 into cannula 314 is established and maintained at the preset level.

The flow valve 320 can be a binary valve, which is in either a fully open or a fully closed position, or, more preferably, a proportional valve, in which the passage is opened different amounts corresponding to various desired flow rates. Also, a normally closed bypass valve 325 is preferably included to circumvent the O₂ flow valve 320 to protect the patient in case of a power failure.

The driver circuit 350 is shown coupled to both O₂ valve 320 and jet pump 310 and can send a signal which initiates adjustment in the size of entrainment apertures (not shown) provided by jet pump 310. The driver circuit 350 thus commands adjustment so that the percentage composition of O₂ output by jet pump 110 is maintained at the predetermined level. For example, if the present invention is connected to a supply of pure O₂ and air through the entrainment port, the predetermined O₂ level is 30%, and the O₂ sensor 330 detects the O₂ level at 25%, the microprocessor could partially close the entrainment valve. The O₂ detector continuously monitors the O₂ level ensuring that it reaches and maintains the targeted level, such as the 30% level.

Several techniques are described below for lowering the resulting delivered 02% to the patient. These techniques can generally be used either alone or in combination with the other techniques presented below.

A low flow resistance cannula arrangement imposes less back pressure on the upstream jet pump. Reduced back pressure allows higher entrainment ratios and a lower resulting delivered 02%. The lower flow resistance can be obtained, for example, by using a larger internal cannula bore, shorter cannula length and different geometries.

Higher effective entrainment ratios and resulting lower delivered 02% may also be obtained using dual or multiple stage air entrainment. In this embodiment, the outflow from an upstream jet pump is used to drive a downstream jet pump.

It may also be possible to filter a portion of the O₂ in the ambient air before mixing in the mixing chamber of the jet pump. Since air includes about 21 volume percent O₂, a reduction in the O₂ percentage in the gas entering the entrainment apertures of the jet pump permits more effective O₂ dilution per volume of entrained gas. For this purpose, a membrane, molecular sieve or zeolite composition can be provided at the entrainment aperture. However, the added flow resistance by placing the membrane or other structure currently available at the entrainment apertures may reduce the entrainment ratio, that is less gas will be entrained for the same flow rate of O₂. The reduction in entrained gas may in some cases substantially negate the otherwise beneficial effect of entraining N₂ vs. air on the delivered O₂%.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

1. An apparatus for delivering a dilute O₂ comprising gas mixture by nasal cannula or face mask to a patient, comprising:
at least one jet pump having an inlet for receiving a high pressure O₂ rich flow and an outlet for emitting a reduced O₂ content flow as compared to said O₂ rich flow, said jet pump having at least one air entrainment aperture, said entrainment aperture entraining room air and providing said reduced O₂ content gas flow at said outlet, and
a nasal cannula or face mask in fluid connection with said outlet of said jet pump, said nasal cannula or face mask including a fluid conduit terminating at a pair of apertured nostril outlet prongs or a face mask for providing said reduced O₂ content gas flow to said patient.

2. The apparatus of claim 1, wherein said jet pump is integrally formed with said nasal cannula or face mask.

3. The apparatus of claim 1, wherein said jet pump is removably coupled to said nasal cannula or face mask, wherein said nasal cannula or face mask is disposable and said jet pump is reusable.

4. The apparatus of claim 1, further comprising structure for adjusting an effective area of said entrainment aperture.

5. The apparatus of claim 1, further comprising a switch disposed between a source of said high pressure O₂ rich flow and said inlet, wherein when said switch is open said jet pump is directly fluidly connected to said source.

6. The apparatus of claim 1, further comprising an O₂ sensor disposed in fluid communication with said outlet, said sensor providing a measured percentage of O₂ in said reduced O₂ content gas flow and generating an electrical output signal based on said measured percentage.

7. The apparatus of claim 6, further comprising interface circuitry including an A/D converter for converting said electrical output signal to a digital signal.

8. The apparatus of claim 7, further comprising a microprocessor-based controller including a memory which stores an O₂ set point for receiving said digital signal, wherein said controller compares said measured percentage to said O₂ set point and generates control signals in response to said comparing.

9. The apparatus of claim 8, further comprising an oxygen valve for controlling a flow of said high pressure O₂ rich flow in series with said input of said jet pump, said driver circuitry being communicably connected to at least one of said jet pump and said oxygen valve, wherein responsive to said control signals said driver circuitry sends adjustment signals to said oxygen valve or said jet pump so that said reduced O₂ content flow tracks said O₂ set point.

10. A method for delivering a dilute O₂ comprising gas mixture by nasal cannula or face mask to a patient, comprising the steps of:
providing a high pressure O₂ rich flow;
entraining room air using said O₂ rich flow using at least one jet pump to provide a reduced O₂ content gas flow, and
delivering said reduced O₂ content gas flow via a nasal cannula or face mask to a patient.

11. The method of claim 10, further comprising the steps of measuring a percentage of O₂ in said reduced O₂ content gas flow, comparing said measured percentage to an O₂ set point, and generating control signals in response to said comparing so that said reduced O₂ content flow tracks said O₂ set point.

12. The method of claim 11, wherein a microprocessor-based controller is used for said comparing and said generating steps.
